(19) United States
(12) Patent Application Publication
(10) Pub. No.: US 2006/0094956 A1
(43) Pub. Date: May 4, 2006

(54) Restricted navigation controller for, and methods of controlling, a remote navigation system

(76) Inventor: Raju R. Viswanathan, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 10/977,466

(22) Filed: Oct. 29, 2004

(51) Int. Cl. A61B 6/00

(52) U.S. Cl. 600/431; 600/434

(57) ABSTRACT

A control and control method for a remote navigation system including an orientation controller that orients the distal end of a medical device in an operating region in a subject, and a length controller that extends and retracts the distal end of the medical device, to navigate the distal end of the medical device to a selected destination in response to user inputs. In accordance with the control method, the control interrupts operation of at least one of the orientation controller and the length controller when the user inputs would cause the orientation controller and the length controller to navigate the distal end of the medical device to one of a plurality of predetermined restricted locations (or alternatively when the user inputs would cause the orientation controller and length controller to navigate the distal end of the medical device other than to one of a plurality of predetermined target locations). In accordance with one preferred embodiment of the method, the control can determine inputs to cause the navigation system to navigate the medical device to an unrestricted location that is closest the user's original selection (or to a target location that is closes to the user's original selection).

### BACKGROUND OF THE INVENTION

An important advance in the field of minimally invasive medical procedures is the development of remote navigation systems that can remotely orient the distal end of a medical device so that the device can be navigated through the body (typically through the subject's vasculature). One type of remote navigation system is a magnetic navigation system such as those disclosed in U.S. Patent Nos. U.S. Pat. No. 6,241,671, issued Jun. 5, 2001, Open Field System for Magnetic Surgery and U.S. patent application Ser. No. 10/056,227, filed Jan. 23, 2002, for Rotating and Pivoting Magnet for Magnetic Navigation (published as 20030137380 on Jul. 23, 2003), the disclosures of which are incorporated by reference. A commercially available magnetic navigation system is the Niobem magnetic surgery system, available from Stereotaxis, Inc., St. Louis, Mo. Other types of remote navigation systems can employ mechanical, electrical, pneumatic, and hydraulic systems for remotely orienting the distal end of a medical device.

A difficulty with remote navigation systems can be controlling the system to quickly and easily navigate the distal end of a medical device to a particular location. Although not necessarily a problem with magnetic navigation systems, sometimes remote navigation systems (such as mechanical systems) must be carefully controlled to prevent the medical device from straying from the intended target or being navigated to locations where the presence of the medical device might cause complications.

### SUMMARY OF THE INVENTION

The present invention provides a controller for, and methods of controlling, a remote navigation system to restrict the possible movements of the medical device. The controller and control methods of the present invention can either restrict the navigation system from navigating the medical device to certain points or sets of points, or restrict the navigation system to navigating the medical device to certain points or sets of points.

Generally, a preferred embodiment of the control of the present invention is adapted for use with a remote navigation system comprising an orientation controller that orients the distal end of a medical device in an operating region in a subject, and a length controller that extends and retracts the distal end of the medical device, to navigate the distal end of the medical device to a target location in response to user inputs. The control interrupts the operation of at least one of the orientation controller and the length controller when the user inputs would cause the orientation controller and the length controller to navigate the distal end of the medical device to one of a plurality of predetermined restricted locations. The control may either simply block further operation of the controllers or alternatively may modify the operation of the medical navigation system to navigate to the closest point that is not in one of the plurality of restricted locations.

In another preferred embodiment, the control overrides the operation of at least one of the orientation controller and the length controller when the user inputs would cause the orientation controller and the length controller to navigate the distal end of the medical device to navigate to a location outside of a plurality of predetermined permitted locations. The control may either simply block further operation of the controllers or alternatively may modify the operation of the medical navigation system to navigate to the closest point within one of the permitted locations.

Thus embodiments of the control and method of the present invention can be employed to prevent a remote navigation system from navigating to selected locations in the operating region. Other embodiments of the control and method of the present invention can be employed to restrict the remote navigation system to navigating to selected locations in the operating region. The restricted navigation permitted by the control and methods of this invention can make operation of a remote navigation system faster and easier. The restricted navigation permitted by the control and methods of this invention can also prevent the medical device from inadvertently being navigated to selected locations or contacting tissue in selected locations. The restricted navigation permitted by the control and methods of this invention also permit the navigation of the device to be restricted to selected locations, facilitating procedures that require repetitive navigation to specified locations. These and other features and advantages will be in part apparent and part pointed out hereinafter.

### BRIEF DESCRIPTION OF THE DRAWINGS

FIG. 1 is a schematic view of one possible interface for a remote navigation system;

FIG. 2 is an enlarged schematic view of an interface showing the identification of restricted and target directions for navigation;

FIG. 3 is an enlarged schematic view of an interface showing the identification of target directions for navigation;

FIG. 4 is an enlarged schematic view of a display of a reconstructed anatomical structure, showing the identification of restricted locations for navigation; and

FIG. 5 shows an interior view of a portion of the Left Atrium where an inclusive region is defined by the closed curve shown therein.

### DETAILED DESCRIPTION OF THE PREFERRED EMBODIMENT

The present invention relates to a control for, and to a method of controlling, a remote medical device navigation system. While shown and described in connection with a magnetic navigation system that magnetically orients and mechanically advances a medical device, the control and method of controlling of this invention is not so limited and can be applied to any remote navigation system, including mechanical systems (such as those use pull wires or push wires to orient the distal end of the device), electrostrictive or piezoelectric systems (such as those using electrostrictive or piezoelectric elements to orient the distal end of the device), pneumatic or hydraulic systems (such as those using fluid pressure to orient the distal end of the device.

A remote medical device navigation system typically includes an orientation controller that orients the distal end of a medical device in an operating region in a subject, and a length controller that extends and retracts the distal end of the medical device. A magnetic medical device navigation system might include an orientation controller that controls the direction of an externally applied magnetic field in the operating region to orient the distal end of a medical device provided with a magnetically responsive element. The magnetic medical device system might also include a length controller, in the form of a device advancer, that advances and retracts the device to change the position of the distal end.

In general a control in accordance with a first preferred embodiment of this invention interrupts the operation of at least one of the orientation controller and the length controller when the user inputs would cause the orientation controller and the length controller to navigate the distal end of the medical device to navigate to one of a plurality of predetermined restricted locations.

The position of the distal end of the device can be determined in several ways. One way of determining the position of the distal end of the device is to use a mathematical model of the medical device with the user inputs as the inputs for the mathematical model. Another way of determining the position of the distal end of the device is to use a localization system to track the position of the distal end of the medical device as the user inputs are applied.

The restricted locations can either be pre-programmed or they can be user defined. If the restricted locations are pre-programmed, the user can be provided with a pick list of sets of restricted locations. Each of these sets can be set up for one or more common procedures. The user can manually select the restricted points or sets of points, for example on a displayed image of the operating region in the subject's body. To facilitate this process the user can be provided with a menu of 2-dimensional and/or 3 dimensional shapes, which the user can select, size, and position on a display.

In an alternate embodiment, the desired restrictions can be defined as restrictions on device tip orientation. For instance, in the preferred embodiment employing a magnetic navigation system, a cone of directions or orientations for the externally applied magnetic field could be defined as an exclusion zone, preventing the device from being oriented within a corresponding range of spatial orientations. As before, the orientation restrictions could be either pre-programmed or user-defined.

The control can operate in a number of ways. In some embodiments, the control may interrupt only the length controller. In other embodiments, the control may interrupt only the orientation controller. In some embodiments, the control can operate the orientation controller as the length controller is operated to change the orientation of the distal end of the medical device so that the distal end of the device does not reach one of the restricted locations. In some embodiments the control can operate the length controller as the orientation controller is operated so that the distal end of the device does not reach one of the restricted locations. In some embodiments, the control can override the user inputs, and generate new inputs to cause the distal end of the medical device to be navigated to the closest location to the user-directed location that is outside of a restricted location. A mathematical model of the medical device is particularly useful for determining the new inputs. The use of a mathematical model of a medical device is taught in U.S. application Ser. No. 10/448,273, filed May 29, 2003 (published as 20040068173 on Apr. 8, 2004), incorporated herein by reference.

In accordance with a second embodiment of the control, rather than preventing the device from being navigated to selected restricted locations, the control can operate the navigation system to confine the device to selected predetermined target locations. In general a control in accordance with a second preferred embodiment of this invention interrupts the operation of at least one of the orientation controller and the length controller when the user inputs would cause the orientation controller and the length controller to navigate the distal end of the medical device to a location outside of one of a plurality of predetermined target locations.

The position of the distal end of the device can be determined in several ways. One way of determining the position of the distal end of the device is to use a mathematical model of the medical device with the user inputs as the inputs for the mathematical model. Another way of determining the position of the distal end of the device is to use a localization system to track the position of the distal end of the medical device as the user inputs are applied.

The target locations can either be pre-programmed or they can be user defined. If the target locations are pre-programmed, the user can be provided with a pick list of sets of target locations. Each of these sets can be set up for one or more common procedures. The user can manually select the target locations, for example on a displayed image of the operating region in the subject's body. To facilitate this process the user can be provided with a menu of 2-dimensional and/or 3 dimensional shapes, which the user can select, size, and position on a display.

In an alternate embodiment, the regions within which it is desired to confine the device can be defined as a range of device tip orientations. For instance, in the case of a magnetic navigation system a cone of directions or orientations for the externally applied magnetic field could be defined as an inclusion zone, preventing the device from being oriented outside of a corresponding range of spatial orientations. As before, the inclusive orientation range could be either pre-programmed or user-defined.

The control can operate in a number of ways. In some embodiments, the control may interrupt only the length controller. In other embodiments, the control may interrupt only the orientation controller. In some embodiments, the control can operate the orientation controller as the length controller is operated to change the orientation of the distal end of the medical device so that the distal end of the device does not reach one of the restricted locations. In some embodiments the control can operate the length controller as the orientation controller is operated so that the distal end of the device does not reach one of the restricted locations. In some embodiments, the control can override the user inputs, and generate new inputs to cause the distal end of the medical device to be navigated to the closest location to the user-directed location that is inside a target location. A mathematical model of the medical device is particularly useful for determining the new inputs.

In accordance with a first preferred embodiment of the method of this invention, the operation of at least one of an orientation controller and a length controller is interrupted when the user inputs to the navigation system would cause the orientation controller and the length controller to navigate the distal end of the medical device to navigate to one of a plurality of predetermined restricted locations.

The position of the distal end of the device can be determined in several ways. One way of determining the position of the distal end of the device is to use a mathematical model of the medical device with the user inputs as the inputs for the mathematical model. Another way of determining the position of the distal end of the device is to use a localization system to track the position of the distal end of the medical device as the user inputs are applied.

The restricted locations can either be pre-programmed or they can be user defined. If the restricted locations are pre-programmed, the user can be provided with a pick list of sets of restricted locations. Each of these sets can be set up for one or more common procedures. The user can manually select the restricted points or sets of points, for example on a displayed image of the operating region in the subject's body. To facilitate this process the user can be provided with a menu of 2-dimensional and/or 3 dimensional shapes, which the user can select, size, and position on a display.

The navigation system can be prevented from navigating to the restricted locations in one of several ways. In some embodiments, the operation of the length controller can be interrupted. In some embodiments, the operation of the orientation controller can be interrupted. In some embodiments, the operation of the orientation controller can be changed as the length controller is operated to change the orientation of the distal end of the medical device so that the distal end of the device does not reach one of the restricted locations. In some embodiments the operation of the length controller can be changed as the orientation controller is operated so that the distal end of the device does not reach one of the restricted locations. In some embodiments, the user inputs can be overridden, and new inputs generated to cause the distal end of the medical device to be navigated to the closest location to the user-directed location that is outside of a restricted location. A mathematical model of the medical device is particularly useful for determining the new inputs.

In accordance with a second preferred embodiment of the method of this invention, rather than preventing the device from being navigated to selected restricted locations, the device can be confined to selected predetermined target locations. In this second embodiment of the method of this invention, the operation of at least one of an orientation controller and a length controller is interrupted when the user inputs to the navigation system would cause the orientation controller and the length controller to navigate the distal end of the medical device to a location outside of one of a plurality of predetermined target locations.

The position of the distal end of the device can be determined in several ways. One way of determining the position of the distal end of the device is to use a mathematical model of the medical device with the user inputs as the inputs for the mathematical model. Another way of determining the position of the distal end of the device is to use a localization system to track the position of the distal end of the medical device as the user inputs are applied.

The target locations can either be pre-programmed or they can be user defined. If the target locations are pre-programmed, the user can be provided with a pick list of sets of target locations. Each of these sets can be set up for one or more common procedures. The user can manually select the target points or sets of points, for example on a displayed image of the operating region in the subject's body. To facilitate this process the user can be provided with a menu of 2-dimensional and/or 3 dimensional shapes, which the user can select, size, and position on a display.

The navigation system can be prevented from navigating outside of the target locations in one of several ways. In some embodiments, the operation of the length controller can be interrupted. In some embodiments, the operation of the orientation controller can be interrupted. In some embodiments, the operation of the orientation controller can be changed as the length controller is operated to change the orientation of the distal end of the medical device so that the distal end of the device does not reach outside of one of the target locations. In some embodiments the operation of the length controller can be changed as the orientation controller is operated so that the distal end of the device does not reach outside of one of the target locations. In some embodiments, the user inputs can be overridden, and new inputs generated to cause the distal end of the medical device to be navigated to the closest location to the user-directed location that is inside one of the target locations. A mathematical model of the medical device is particularly useful for determining the new inputs.

In one particular preferred embodiment, the orientation of the distal end of the medical device is estimated if user inputs were applied, using a mathematical model of the medical device. The operation of the medical navigation system is then modified if the estimated orientation of the distal end of the medical device is not within a predetermined angle from a predetermined path at the current location.

In another particular embodiment the position of the distal end of the medical device is input as if user inputs were applied, using a mathematical model of the medical device. The operation of the medical navigation system is modified to inhibit advancement of the distal end of the medical device if the estimated position of the distal end of the medical device is within a predetermined distance from at least one predetermined restricted location. In an alternate embodiment, the operation of the medical navigation system is modified to override the user-controlled speed of advancement of the distal end of the medical device if the estimated position of the distal end of the medical device is within a predetermined distance from at least one predetermined location.

According to some embodiments of the methods of this invention, the position of the distal end of the medical device if the inputs were applied is estimated using a mathematical model of the medical device. The operation of the medical navigation system can be modified in response, for example to change the orientation of the medical device as the device is advanced to prevent the distal end of the device from reaching a predetermined restricted location. The operation is preferably modified so that so that the navigation system positions the distal end of the medical device outside of the restricted location at a point closest to the location corresponding to the user's original inputs. Of course rather than modifying the operation to prevent the device from reaching a restricted location, the method can instead modify the operation to cause the device to reach a target location. In particular, the navigation system can be adjusted so that the navigation system positions the distal end of the medical device inside of the target location closest to the location corresponding to the user's original inputs. This can be done by determining substitute inputs for the user inputs, for example using a mathematical model.

Of course, rather than estimating the position, localization information can be used to sense the actual position of the distal end of the medical device as the navigation system operates. In this case, the operation of the medical device navigation system can be altered if the sensed position is within a predetermined distance of the at least one restricted location, to prevent the device from reaching the at least one restricted location. Alternatively the operation of the medical device navigation system can be altered if the sensed position is not within a predetermined distance of the at least one predetermined target location to cause medical navigation device to reach one of the predetermined target locations.

In an alternate embodiment, the regions within which it is desired to either confine the device or exclude it, while the advancement or retraction is in effect, can be defined as a range of device tip orientations. In this manner, the exclusion or inclusion zone can be defined even in the absence of a mathematical model or when device localization inputs are not available. For instance, in the case of a magnetic navigation system a cone of directions or orientations for the externally applied magnetic field could be defined as an inclusion zone, preventing the device from being oriented outside of a corresponding range of spatial orientations. As before, the inclusive orientation range could be either pre-programmed or user-defined. Likewise, exclusion zones based on a range of orientations could be implemented. Restriction based on device tip orientation can be easily implemented using either mathematical models of the device or device localization, which can detect the current position and orientation of the device. With device localization, when a restricted direction is detected, the length controller can be inhibited and/or the orientation controller can be operated to change the orientation of the device.

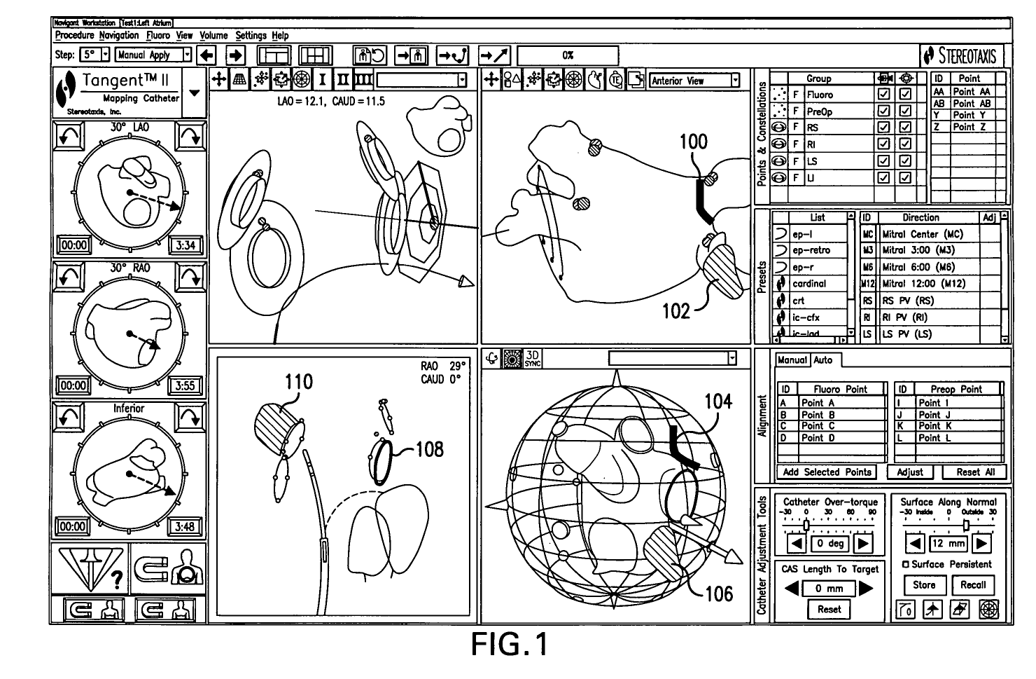

An example of a display from a graphical user interface for controlling a magnetic navigation system is shown in FIG. 1. Examples of other interfaces for controlling remote navigation systems are disclosed in U.S. patent application Ser. No. 10/844,055, filed May 12, 2004, based upon PCT Application No. PCT/US03/22919, filed Jul. 22, 2003; U.S. patent application Ser. No. 10/942,748, filed Sep. 16, 2004; U.S. patent application Ser. No. 10/448,273, filed May 29, 2003. U.S. Provisional Patent Application Ser. No. 60/576,946, filed Jun. 4, 2004 U.S. patent application Ser. No. 10/942,748, filed Sep. 16, 2004, the disclosures of each of which are incorporated herein by reference. The display includes a number of tools for identifying locations and directions to the magnetic navigation system. One of these tools is an anatomical model of the objects in the operating region in the subject. In accordance with the controls and control methods of this invention, the user can identify a restricted area or volume into which the user does not wish to navigate the distal end of the medical device or the user can identify a target area or volume into which the user wants to confine the distal end of the medical device. Using conventional drawing tools, similar to the drawing tools available in most office programs such as Microsoft Word or Microsoft Power Point or Microsoft Visio, the user can identify points, lines, loops, or shapes corresponding to restricting locations or to the target locations. For example, on the object interface, the user can identify a line 100 on the surface of the anatomy in the operating region to which navigation should be restricted. The user can alternatively, or in addition, also identify an area 102 on the surface of the anatomy in the operating region from which navigation should be restricted.

In FIG. 1 there is also a tool for selecting the direction of orientation of the medical device, in the form of the sphere. The user can identify points, lines, loops and shapes to on this sphere, and the control can restrict the navigation system from aligning the medical device in the corresponding directions, or the control can restrict the navigation system to aligning the medical device in the corresponding directions. For example, on the object interface, the user can identify a line 104 or a loop 106 on the surface of the sphere corresponding to directions in the operating region to which navigation should be restricted. The user can alternatively, or in addition, also identify an area 108 on the surface of the sphere corresponding to directions in the operating region from which navigation should be restricted.

In FIG. 1 there is also a tool for locations in the operating region directly on a medical image of the operating region. In this case the image is a two-dimensional x-ray image, but the image could also some other two-dimensional or three-dimensional imaging. The user can identify points, lines, loops and shapes in the operating region on the display, and the control can restrict the navigation system from navigating the medical device to the corresponding locations, or the control can restrict the navigation system to navigating to the corresponding locations. For example in the image of the operating region, the user can identify a target loop 110 to which the user wants to restrict navigation of the medical device, for example to make a closed loop of ablations to isolate an aberrant electrical signal. Similar the user can identify an area 112 from which the user wants to restrict navigation of the medical device. The control can operate the navigation system in response to user inputs to restrict navigation to the loop 110 or to restrict navigation from the area 112.

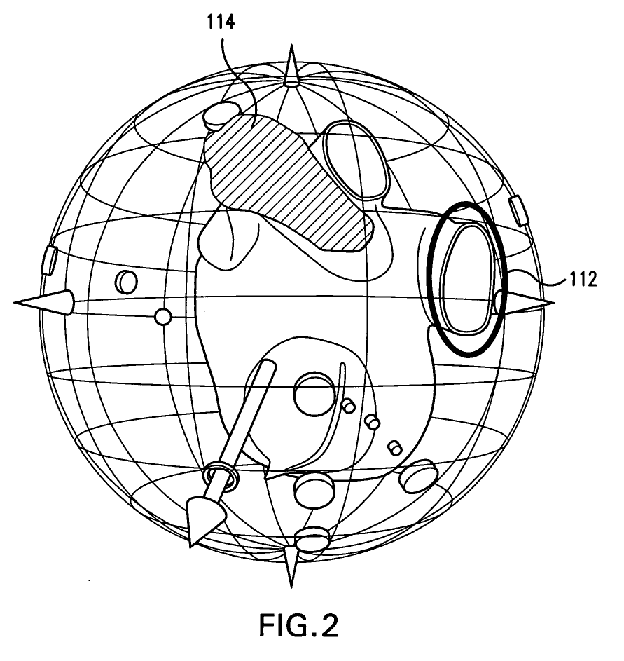

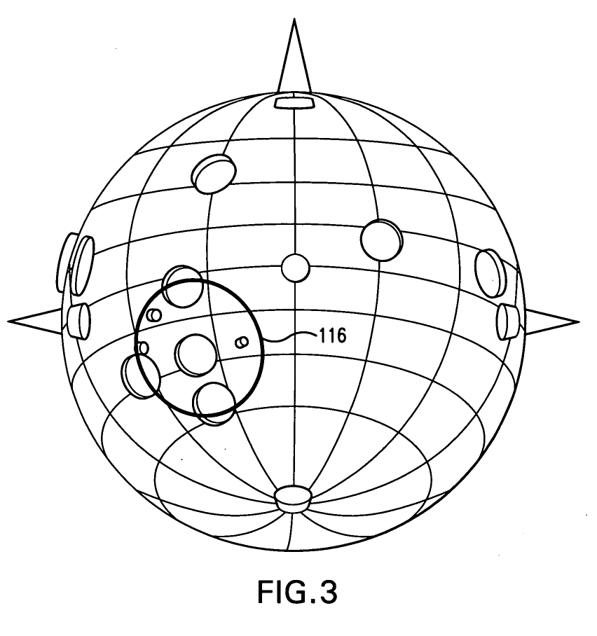

The spherical interface is shown in FIG. 1 greater detail in FIGS. 2 and 3. In FIG. 2, the interface includes a representation of the anatomy in the operating region and a spherical object for identifying directions to apply magnetic fields and therefore orient the distal end of the medical device. As shown in FIG. 2 the user can identify points, lines, loops, and shapes on the surface corresponding to directions that the navigation system is restricted from navigating to, or restricted to navigating to. For example, the user can position a loop 114 on the sphere to identify directions to which the navigation is restricted. The user a also position an area 116 on the sphere to identify directions from which navigation is restricted.

FIG. 3 is an alternate view of the interface in which the inside of the spherical object is visible. Various landmark are indicated on the surface, that the user can use to position restricted navigation zones. For example, using the markers for the mitral valve, the user can construct a loop 118 on the sphere around the mitral valve corresponding to the directions required to navigate around the mitral valve. By restricting the navigation to directions on this loop, the user can quickly and easily navigate the distal end of the device to contact points in a closed loop around a structure, such as the mitral valve

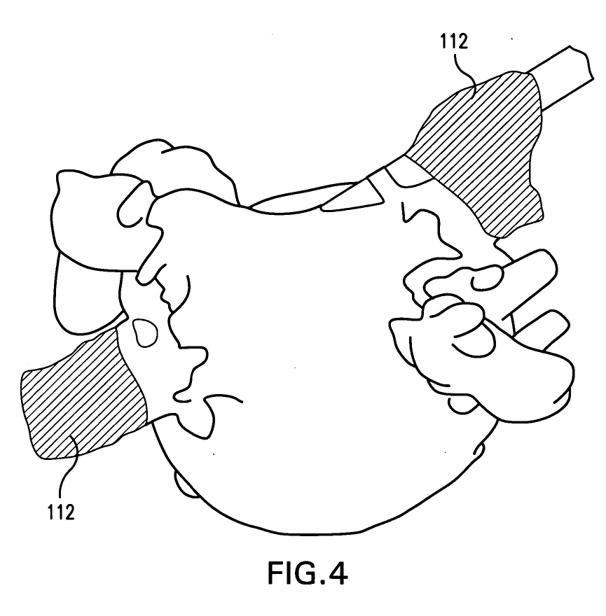

As shown in FIG. 4, the interface can also provide a detailed reconstruction of an anatomical feature in the operating region in the subject, and the user can identify points, lines, loops, and shapes on the surface corresponding to locations to which the user confines navigation, or from which the user restricts navigation. As shown in FIG. 4, two such areas 120 and 122 have been identified, representing locations on the surface of the corresponding anatomical structure in the operating region (e.g., the heart) to which the user does not wish to navigate the distal end of the device.

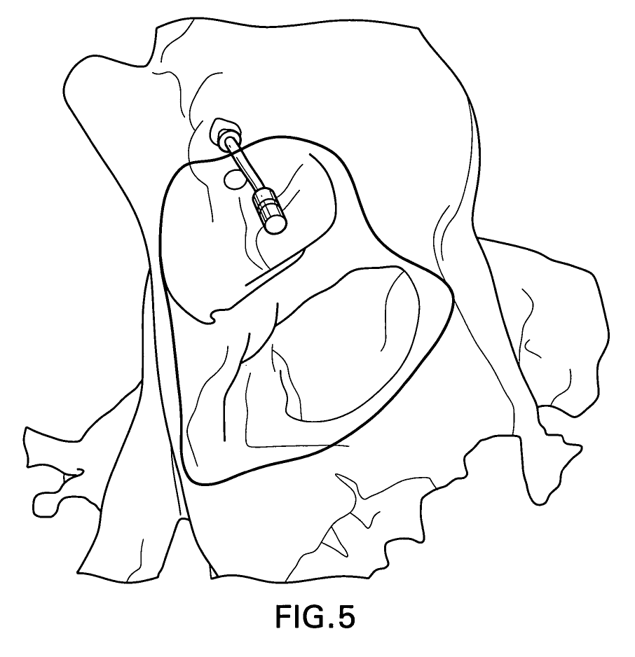

FIG. 5 provides an interior view of a portion of the Left Atrium where a closed curve depicts the region on the wall that is defined as an inclusive zone within which it is desired to maintain the catheter. This spatial zone could be translated into an equivalent restriction on control variables that actuate the catheter. A visual display of the tip of a localized catheter is also shown therein, as well as a target location on the wall. In the case when a magnetic navigation system actuates the catheter, the external field direction could also be displayed.

Thus, in an electrophysiology mapping procedure, the user can specify locations where the user wants to map electrical activity and constrain navigation to those points to make navigation quicker and easier. Similarly in an electrophysiology ablation procedure, the user can specify locations wherein the user either wants to ablate tissue, or does not want to ablate tissue, and constrain the navigation to (or from) those points.

What is claimed is:

1. A control for a remote navigation system including an orientation controller that orients the distal end of a medical device in an operating region in a subject, and a length controller that extends and retracts the distal end of the medical device, to navigate the distal end of the medical device to a selected destination in response to user inputs, the control interrupting operation of at least one of the orientation controller and the length controller when the user inputs would cause the orientation controller and the length controller to navigate the distal end of the medical device to one of a plurality of predetermined restricted locations.

2. The control according to claim 1 wherein at least some of the predetermined restricted locations are pre-programmed.

3. The control according to claim 1 wherein at least some of the predetermined locations are input by the user.

4. The control according to claim 1 wherein the control interrupts only the length controller.

5. The control according to claim 1 wherein the control interrupts only the orientation controller.

6. The control according to claim 1 wherein the control operates the orientation controller as the length controller is operated to change the orientation of the distal end of the medical device so that the distal end of the device does not reach one of the predetermined restricted locations.

7. The control according to claim 1 wherein the control operates the length controller as the orientation controller is operated so that the distal end of the device does not reach one of the predetermined restricted locations.

8. A control for a remote navigation system including an orientation controller that orients the distal end of a medical device in an operating region in a subject, and a length controller that extends and retracts the distal end of the medical device, to navigate the distal end of the medical device to a selected destination in response to user inputs, the control overriding the operation of at least one of the orientation controller and the length controller when the user inputs would cause the orientation controller and the length controller to navigate the distal end of the medical device to a location outside of a plurality of predetermined target locations.

9. The control according to claim 8 wherein at least some of the predetermined target locations are pre-programmed.

10. The control according to claim 8 wherein at least some of the predetermined target locations are input by the user.

11. The control according to claim 8 wherein the control interrupts only the length controller.

12. The control according to claim 8 wherein the control interrupts only the orientation controller.

13. The control according to claim 8 wherein the control operates the orientation controller as the length controller is operated to change the orientation of the distal end of the medical device so that the distal end of the device only reaches the predetermined target locations.

14. A method for operating a remote navigation system including an orientation controller that orients the distal end of a medical device in an operating region in a subject, and a length controller that extends and retracts the distal end of the medical device, to navigate the distal end of the medical device to a selected destination in response to user inputs, the control interrupting operating of at least one of the orientation controller and the length controller when the user inputs would cause the orientation controller and the length controller to navigate the distal end of the medical device to one of a plurality of predetermined restricted locations.

15. The method according to claim 14 wherein at least some of the predetermined restricted locations are pre-programmed.

16. The method according to claim 14 wherein at least some of the predetermined restricted locations are input by the user.

17. The method according to claim 14 wherein only the length controller is interrupted.

18. The method according to claim 14 wherein only the orientation controller is interrupted.

19. The method according to claim 14 wherein the operation of the orientation controller is altered as the length controller is operated to change the orientation of the distal end of the medical device so that the distal end of the device does not reach one of the restricted locations.

20. The method according to claim 14 wherein the operation of the length controller is altered as the orientation controller is operated so that the distal end of the device does not reach one of the restricted locations.

21. A method for operating a remote navigation system including an orientation controller that orients the distal end of a medical device in an operating region in a subject, and a length controller that extends and retracts the distal end of the medical device, to navigate the distal end of the medical device to a selected destination in response to user inputs, the control overriding the operation of at least one of the orientation controller and the length controller when the user inputs would cause the orientation controller and the length controller to navigate the distal end of the medical device to navigate to a location outside of a plurality of predetermined target locations.

22. The method according to claim 21 wherein at least some of the predetermined target locations are pre-programmed.

23. The method according to claim 21 wherein at least some of the predetermined target locations are input by the user.

24. The method according to claim 21 wherein only the length controller is interrupted.

25. The control according to claim 21 wherein only the orientation controller is interrupted.

26. The control according to claim 21 wherein the operation of the orientation controller is altered as the length controller is operated to change the orientation of the distal end of the medical device so that the distal end of the device only reaches one of the plurality of predetermined target locations.

27. A method for operating a remote navigation system including an orientation controller that orients the distal end of a medical device in an operating region in a subject, and a length controller that extends and retracts the distal end of the medical device, the method including modifying the operation of the orientation controller to maintain the distal end of the medical device within a predetermined range target of orientations.

28. The method of claim 27 wherein the predetermined range of target orientations is pre-programmed.

29. The method of claim 27 wherein the predetermined range of target orientations is input by a user.

30. A method for operating a remote navigation system including an orientation controller that orients the distal end of a medical device in an operating region in a subject, and a length controller that extends and retracts the distal end of the medical device, the method comprising restricting the operation of the orientation controller to cause the distal end of the medical device to remain oriented outside of a restricted range of orientations.

31. The method of claim 30 wherein the restricted range of orientations is pre-programmed.

32. The method of claim 30 wherein the restricted range of orientations is input by a user.

33. A method for governing the operation of medical navigation system which orients the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system if the estimated position of the distal end of the medical device is at one of a predetermined number of restricted locations so that the medical navigation system does not navigate the distal end of the medical device to one of the predetermined restricted locations.

34. A method for governing the operation of medical navigation system which orients the distal end of a medical device in an operating region in a subject in a selected direction in response to user inputs, the method comprising:
accepting input of current device tip location from a localization system;
modifying the operation of the medical navigation system if the current device tip location is within a specified distance of at least one of a plurality of specified locations in order to prevent navigation of the device to any one of said plurality of specified locations.

35. A method for governing the operation of medical navigation system which orients the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system if the estimated position of the distal end of the medical device is not at one of a predetermined number of target locations so that the medical navigation system navigates the distal end of the medical device to one of the predetermined target locations.

36. A method for governing the operation of medical navigation system which orients the distal end of a medical device in an operating region in a subject in a selected direction in response to user inputs, the method comprising:
accepting input of current device tip location from a localization system;
modifying the operation of the medical navigation system if the current device tip location is within a specified distance of at least one of a plurality of specified locations which define a region in order to prevent navigation of the device to locations outside the region.

37. A method for governing the operation of medical navigation system which orients the distal end of a medical device in an operating region in a subject in a selected direction in response to user inputs, the method comprising:
estimating the orientation of the distal end of the medical device if the inputs were applied, using a mathematical model of the medical device;
modifying the operation of the medical navigation system if the estimated orientation of the distal end of the medical device is not within a predetermined angle from a predetermined path at the current location so that the medical navigation system does not orient the distal end of the medical device at an angle greater than a predetermined error from the predetermined path.

38. A method for governing the operation of medical navigation system which orients and advances the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system to inhibit advancement of the distal end of the medical device if the estimated position of the distal end of the medical device is within a predetermined distance from at least one predetermined restricted location.

39. A method for governing the operation of medical navigation system which orients and advances at a user-controlled speed, the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system to override the user-controlled speed of advancement of the distal end of the medical device if the estimated position of the distal end of the medical device is within a predetermined distance from at least one predetermined location.

40. A method for governing the operation of medical navigation system which orients and advances the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system to change the orientation of the medical device as the device is advanced to prevent the distal end of the device from reaching at least one predetermined restricted location.

41. The method according to claim 40 wherein the medical navigation system comprises an orientation system for orienting the distal end of the medical device, and a motion system for changing the length of the distal end of the medical device, and wherein the step of modifying the operation of the medical navigation system comprises adjusting the orientation system so that the navigation system positioned the distal end of the medical device outside of the restricted locations closest to the location corresponding to the user's original inputs.

42. The method according to claim 40 wherein the medical navigation system comprises an orientation system for orienting the distal end of the medical device, and a motion system for changing the length of the distal end of the medical device, and wherein the step of modifying the operation of the medical navigation system comprises limiting the operation of the motion system to prevent the distal end from reaching at least one predetermined location.

43. The method according to claim 40 wherein the medical navigation system comprises an orientation system for orienting the distal end of the medical device, and a motion system for changing the length of the distal end of the medical device, and wherein the step of modifying the operation of the medical navigation system comprises limiting the speed of operation of the motion system in proximity to at least one predetermined location.

44. A method for governing the operation of medical navigation system which orients and advances the distal end of a medical device in an operating region in a subject in a selected direction in response to user inputs, the method comprising:
estimating the position of the distal end of the medical device if the inputs were applied, using a mathematical model of the medical device;
modifying the operation of the medical navigation system to change the orientation of the medical device as the device is advanced to cause the distal end of the device to reach at least one predetermined target location.

45. The method according to claim 44 wherein the medical navigation system comprises an orientation system for orienting the distal end of the medical device, and a motion system for changing the length of the distal end of the medical device, and wherein the step of modifying the operation of the medical navigation system comprises adjusting the orientation system so that the navigation system positions the distal end of the medical device inside of the target location closest to the location corresponding to the user's original inputs.

46. A method for governing the operation of medical navigation system which orients and advances the distal end of a medical device in an operating region in a subject in a selected direction in response to user inputs, the method comprising:
accepting input of current device tip location and orientation from a localization system;
modifying the operation of the medical navigation system if the current device tip orientation is within a specified range of orientations to prevent navigation of the device to selected locations.

47. A method for governing the operation of medical navigation system which orients and advances the distal end of a medical device in an operating region in a subject in a selected direction in response to user inputs, the method comprising:
accepting input of current device tip location and orientation from a localization system;
modifying the operation of the medical navigation system if the current device tip orientation is within a specified range of orientations to restrict navigation of the device to selected locations.

48. A method of governing the operation of a medical navigation system for orienting and advancing the distal end of a medical device in response to control inputs from a user, the method comprising:
accepting the input of at least one restricted location;
estimating the position of the distal end of the medical device if the control inputs were applied, using a mathematical model of the medical device;
modifying the control inputs if the estimated position coincides with the at least one restricted location, to cause medical navigation system to orient and advance the medical device to a position other than the at least one restricted location.

49. The method according to claim 38 wherein the step of modifying the control inputs comprises determining an alternate location, and using a mathematical model of the medical device determining inputs to the navigation system to cause it to navigate the distal end of the medical device to the alternate location.

50. The method according to claim 38 wherein the step of accepting the input of at least one restricted location comprises accepting a selection of one of a plurality of predetermined sets of restricted locations.

51. A method of governing the operation of a medical navigation system for orienting and advancing the distal end of a medical device in response to control inputs from a user, the method comprising:
accepting the input of at least one restricted location;
estimating the position of the distal end of the medical device if the control inputs were applied, using a mathematical model of the medical device;
modifying the control inputs if the estimated position does not coincide with the at least one predetermined target location, to cause medical navigation system to orient and advance the medical device to a position in one of the predetermined target locations.

52. The method according to claim 41 wherein the step of modifying the control inputs comprises determining an alternate location, and using a mathematical model of the medical device determining inputs to the navigation system to cause it to navigate the distal end of the medical device to the alternate location.

53. The method according to claim 41 wherein the step of accepting the input of at least one target location comprises accepting a selection of one of a plurality of predetermined sets of target locations.

54. A method of governing the operation of a medical navigation system for orienting and advancing the distal end of a medical device in response to control inputs from a user, the method comprising:
accepting the input of at least one restricted location;
estimating the position of the distal end of the medical device if the control inputs were applied, using a mathematical model of the medical device;
restricting the operation of the medical navigation system to prevent the distal end of the medical device from reaching the at least one restricted location.

55. A method of governing the operation of a medical navigation system for orienting and advancing the distal end of a medical device in response to control inputs from a user, the method comprising:
accepting the input of at least one restricted location;
estimating the position of the distal end of the medical device if the control inputs were applied, using a mathematical model of the medical device;
restricting the operation of the medical navigation system to cause the distal end of the medical device to reach only predetermined target locations.

56. A method of governing the operation of a medical device navigation system for orienting and advancing the distal end of a medical device in response to control inputs from a user, the method comprising:
sensing the current position of the distal end of the medical device;
modifying the control inputs if the sensed position is within a predetermined distance of at least one restricted location, to cause medical navigation system to orient and advance the medical device to a position other than the at least one restricted location.

57. A method of governing the operation of a medical device navigation system for orienting and advancing the distal end of a medical device in response to control inputs from a user, the method comprising:
sensing the current position of the distal end of the medical device;
modifying the control inputs if the sensed position is not within a predetermined distance of at least one of a plurality of predetermined target locations, to cause medical navigation system to orient and advance the medical device to one of a plurality of predetermined target locations.

58. A method of governing the operation of a medical device navigation system for orienting and advancing the distal end of a medical device in response to control inputs from a user, the method comprising:
sensing the current position of the distal end of the medical device;
altering the operation of the medical device navigation system if the sensed position is within a predetermined distance of the at least one restricted location, to prevent the device from reaching the at least one restricted location.

59. A method of governing the operation of a medical device navigation system for orienting and advancing the distal end of a medical device in response to control inputs from a user, the method comprising:
sensing the current position of the distal end of the medical device;
altering the operation of the medical device navigation system if the sensed position is not within a predetermined distance of the at least one predetermined target location to cause medical navigation device to reach one of the predetermined target locations.
